(19) United States
(12) Patent Application Publication
(10) Pub. No.: US 2008/0033422 A1
(43) Pub. Date: Feb. 7, 2008

(54) Microwave applicator with margin temperature sensing element

(76) Inventors: Paul F. Turner, Bountiful, UT (US); Theron N. Schaefermeyer, North Salt Lake, UT (US); Thomas L. Youd, Salt Lake City, UT (US)

Correspondence Address: THORPE NORTH & WESTERN, LLP., 8180 SOUTH 700 EAST, SUITE 350, SANDY, UT, 84070, US

(21) Appl. No.: 11/499,079

(22) Filed: Aug. 4, 2006

(51) Int. Cl. A61B 18/04; A61F 2/00; A61N 1/00

(52) U.S. Cl. 606/33; 607/102; 607/156

(57) ABSTRACT

A microwave applicator for applying microwave radiation to body tissue includes a temperature sensor positioned along the applicator to measure the temperature of body tissue at a margin of the tissue to be treated. By monitoring the temperature of the tissue at the margin of the tissue to be treated, the heating of the tissue can be better controlled to ensure that the tissue to be treated is heated to the required temperature while damage to surrounding normal tissue is minimized. Treatment can include positioning one or more applicators into body tissue and applying microwave radiation to the applicators. Phase and amplitude control of the microwave radiation can be used to produce a desired heating pattern. Optimization of the number and location of microwave applicators and the phase and amplitude of microwave energy applied thereto can be determined through pretreatment simulation.

### BACKGROUND OF THE INVENTION

1. Field

This invention relates to electromagnetic radiation (EMR) therapy and more particularly to applicators for applying electromagnetic energy to a treatment site to heat the treatment site.

2. State of the Art

The use of electromagnetic (EM) energy to heat tissue for the treatment of disease is known. For example, death, or necrosis, of living tissue cells occurs at temperatures elevated above a normal cell temperature. Above a threshold temperature of about 41.5 degrees C., substantial thermal damage occurs in most malignant cells. At temperatures above about 45 degrees C. thermal damage occurs to most normal cells when exposed for more than 30 minutes. The death rate of heated tissue cells is a function of both the temperature to which the tissue is heated and the duration for which the tissue is held at such temperatures. Thermal dose has been generally accepted for cancer treatments as the equivalent number of minutes of exposure as though the tissue had been at 43 degrees C. This means that if a tumor had been at 43 degrees C. for 30 minutes it would have an equivalent thermal dose of 30 minutes, usually referred to as a thermal dose of 30. For temperatures above 43 degrees C., each additional degree C. in temperature effectively doubles the thermal dose. Hence, a treatment at 50 degrees C. will have 128 times the thermal dose of treatment at 43 degrees C. for a given time interval. During treatment, it is desirable to produce an elevated temperature within the targeted tissue, while keeping nearby healthy tissue at a safe lower temperature. For this reason, when treatment methods are used which can provide adequate thermal damage to destroy a cancerous tumor with heat alone while adequately protecting the surrounding normal tissues, very high tumor temperatures are typically used. In such conditions it is important to assure both adequate tumor heating at the tumor margin and reduced temperatures in the critical normal tissues.

Heating therapy is sometimes combined with other treatments, such as surgery, ionizing radiation, and chemotherapy. For example, when heating is combined with radiation, it is desirable to maintain the temperature within the diseased tissue within the range of about 42 to 45 degrees C. Higher temperatures are usually undesirable when a combined treatment modality is used because higher temperatures can lead to microvessal collapse causing resistance to radiation therapy and decrease the amount of systemic chemotherapy from reaching the tumor if it has vascular damage. Lower temperatures are also undesirable because they can fail to provide adequate therapeutic effect. Therefore, it is important to control the temperature within the desired range for multi-modality treatments and not allow heating of the tissue in the tumor or around the tumor to above 45 degrees C. if such tissue damage from other treatments may be compromised. Since with prior art electromagnetic energy applicators the center portion of a tumor will generally reach the highest temperature, where a temperature sensor has been used as part of the EM applicator, the temperature sensor has been located to measure the temperature in the center of the heated tissue area so that the maximum temperature of the heated tissue can be measured and controlled. At times, in such conditions, the highest tissue temperature may be the limiting factor in heating the tissue. The goal is to heat all the tumor sufficiently while not excessively heating the tumor.

Alternate forms of thermal therapy kill the tissue with heating alone. However, to adequately eradicate a cancerous tumor with only the application of heat, it is necessary to assure adequate heating is accomplished throughout the tumor. In cases of a malignant tumor, if viable tumor cells are left behind, the tumor can rapidly grow back leaving the patient with the original problem. It is generally recognized that to eradicate a tumor by heating, a thermal dose of at least 200 throughout the target tumor should be applied. If the thermal dose within the entire volume of the tumor exceeds this range significantly, it is quite certain that the tumor will be completely eradicated. One alternate form of thermal therapy is microwave ablation, where diseased tissue is heated to temperatures sufficient to kill the diseased tissue. Temperatures used in ablation usually reach 60 degrees C. or higher. In ablation therapy it is less important to maintain an elevated temperature within the diseased tissue (provided adequately high temperatures are reached to produce the desired therapeutic effect) than in treatments where the maximum temperature of the tissue has to be controlled. However, with heat ablation treatments, heating treated tissue to 60 degrees C. or above, there is a volume reduction of temperature that ranges from this high temperature in the treated tissue to the normal tissue temperature of 37 degrees C. outside the treated tissue. The outer margin of the overall heat distribution in this tissue volume may then result in damage to normal tissue if such normal tissue is exposed to a thermal dose level that reaches 200 equivalent minutes. Therefore, for prolonged ablation treatments where the ablation volume is maintained at very high temperatures there is a high risk of damage to surrounding normal tissues. For proper treatment of such targeted cancerous tumor volumes, it becomes very important to properly deliver the correct thermal distribution over a sufficient time period to eradicate the tumor tissue while minimizing damage to critical surrounding normal tissue. Fortunately, there are tumor locations that reside in normal tissue that can be destroyed by the heating in limited areas without affecting the health of the patient, such as liver tissue. In such situations the ablation can be applied in an aggressive way to include a margin of safety in destruction of limited surrounding normal tissues to assure that all the cancerous tumor is destroyed.

The process of heating very rapidly to high temperatures that is common in ablation treatments may utilize a rather short exposure time. In doing so, the resulting temperature distribution becomes primarily a result of the power absorption distribution within the tissue. However, if such treatments continue for multiple minutes, the blood flow and thermal conduction of the tumor and surrounding tissues will modify the temperature distribution to result in a less predictable heat distribution because the changes occurring in bloodflow in such a heated region may not be predictable. Therefore, it is important to optimize the uniformity of the tissue heating power that is absorbed to lead to a more predictable temperature distribution that better corresponds with the treatment prescription. In the temperature ranges of thermal therapy and hyperthermia where lower temperatures are used, typically between 40 and 60 degrees C., the importance of optimizing the temperature distribution and power distribution is also important. Therefore, pretreatment planning practices prior to and possibly during treatment for calculating the power and temperature distribution resulting from the parameters of power and relative phase of the power applied to the tissue could be important for both ablation as well as thermal therapy and hyperthermia. As temperatures are higher during treatment it may increase patient discomfort and pain, so it can be helpful to avoid excessive temperatures to reduce the need of patient sedation.

Invasive microwave energy applicators can be inserted into living body tissue to place the source of heating into or adjacent to a diseased tissue area. Invasive applicators help to overcome some difficulties that surface applicators experience when the target tissue region is located below the skin (e.g., the prostrate). Invasive applicators must be properly placed to localize the heating to the vicinity of the desired treatment area. Even when properly placed, however, it has been difficult to ensure that adequate heat is developed in the diseased tissue without overheating surrounding healthy tissue.

### SUMMARY OF THE INVENTION

According to the invention, a microwave applicator for applying microwave radiation to body tissue includes a temperature sensor positioned along the applicator to measure the temperature of body tissue at a margin of the tissue to be treated. By monitoring the temperature of the tissue at the margin of the tissue to be treated, the heating of the tissue can be better controlled to ensure that the tissue to be treated is heated to the required temperature while damage to surrounding normal tissue is minimized. The control of the heating may further include the systematic use of such applicators in phased arrays with optimization computational guidance in the form of pretreatment planning to provide an ideal insertion pattern and power and phase application to the array of applicators to produce and control uniform temperatures throughout the tumor volume, and particularly at the tumor margins. The treatment is thereby optimized and controlled by the adjustment of power amplitude and phase of each of the inserted applicators as directed by a computer-controlled system using the integrated temperature sensors positioned at the heating region margins.

One embodiment of the present invention includes a microwave applicator for heat treatment of diseased tissue within a living body. The applicator includes an elongate applicator body having a proximal end for insertion into a tissue region of the living body and a distal end for attachment to a source of microwave energy. An antenna is disposed toward the proximal end of the applicator body. Microwave energy is conducted from the distal end to the antenna via a microwave energy conductor disposed within the applicator body. A temperature sensor is positioned along the applicator body to place the temperature sensor at a position corresponding to an outer margin of an expected heating area in the living body tissue caused by the antenna during operation of the applicator.

### THE DRAWINGS

Other features of the invention will become more readily apparent from the following detailed description when read in conjunction with the drawings in which the accompanying drawings show the best modes currently contemplated for carrying out the invention, and wherein:

FIG. 1 is a cross sectional view of an applicator, in accordance with an embodiment of the present invention;

FIG. 2 is a cross sectional view of the applicator of FIG. 1 showing uniform temperature contours when the applicator is inserted into living body tissue;

FIG. 3 is a cross section view of an alternate embodiment of an applicator having a sleeve in accordance with an embodiment of the present invention;

FIG. 4 is a cross sectional view of an applicator sheathed by a closed end catheter, in accordance with an embodiment of the present invention;

FIG. 5 is a cross sectional view of an applicator sheathed by a hollow needle, in accordance with an embodiment of the present invention;

FIG. 6 is a block diagram of a system for microwave therapy for heat treatment of diseased tissue within a living body, in accordance with an embodiment of the present invention;

FIG. 7 is a screen capture of an exemplary pretreatment planning system, in accordance with an embodiment of the present invention;

FIGS. 8-11 are screen captures of the pretreatment planning system displaying two-dimensional slices of simulated heating at various positions in a third dimension, where the phase of microwave energy applied to the microwave applicators is non-coherent; and

FIGS. 12-15 are screen captures displaying heating for the same area of FIGS. 8-11, where the phase and amplitude of the microwave energy is optimized to maximize uniformity of the heating.

### DETAILED DESCRIPTION OF THE ILLUSTRATED EMBODIMENTS

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

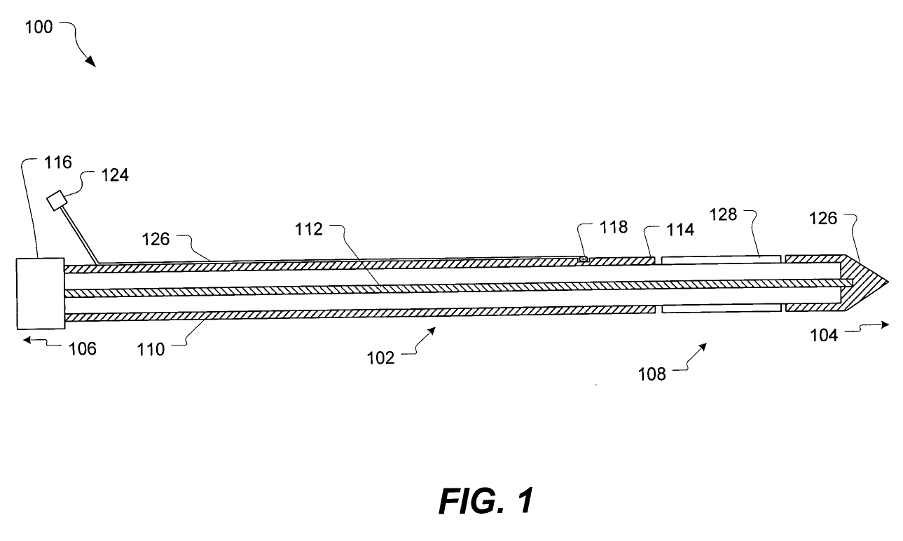

A microwave applicator for heat treatment of diseased tissue within a living body tissue is illustrated in FIG. 1. The applicator, shown generally at 100, includes an elongate applicator body 102 having a proximal end 104 for insertion into a tissue region of the living body and a distal end 106 for attachment to a source of microwave energy. A means for radiating microwave energy, for example, an antenna 108, is disposed at the proximal end of the applicator body. Microwave energy includes electromagnetic (EM) energy, such as a traveling EM or radiated EM wave having a frequency greater than about 300 MHz, including for example, 915 MHz.

A means for conducting microwave energy is disposed within the applicator body 102 to conduct microwave energy from the distal end of the applicator body to the means for radiating. For example, the means for conducting microwave energy can be provided by a coaxial transmission line. The coaxial transmission line can be formed by a conductive shell 110 portion of the applicator body which functions as an outer conductor of the coaxial transmission line and a center conductor 112 disposed within the applicator body. A connector 116 can be provided at the distal end for coupling microwave energy into the applicator. Alternately, the means for conducting microwave energy can include a cable attached to the proximal end 106 of the microwave applicator 100, the cable extending some distance to a connector.

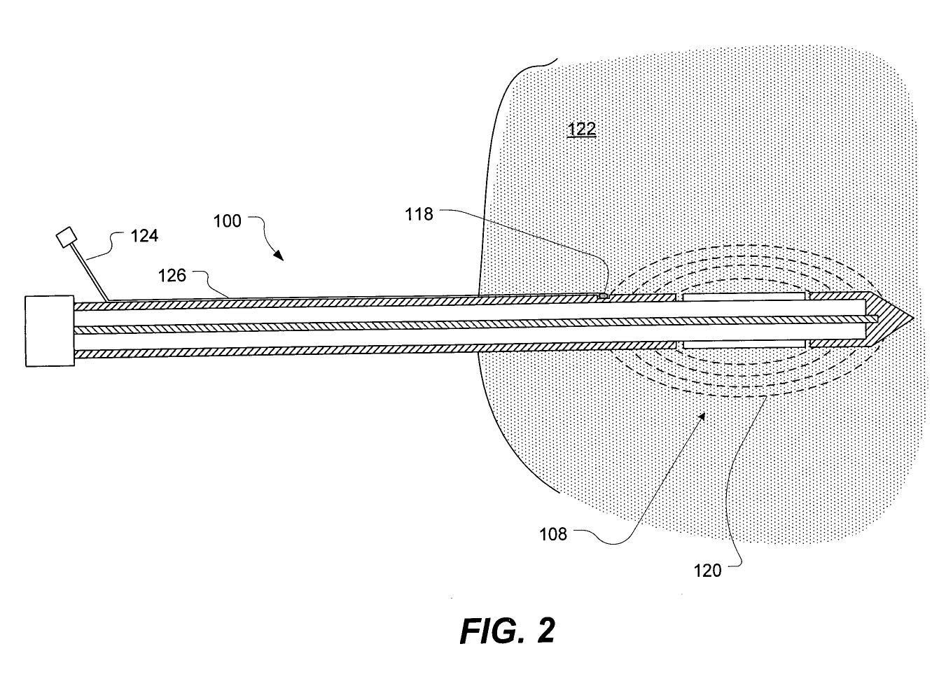

The microwave applicator 100 also includes a means for sensing temperature, such as a temperature sensor 118. The means for sensing temperature is positioned along the applicator body at a point corresponding to an outer margin of an expected heating area in the living body tissue. For example, FIG. 2 provides an illustration of uniform temperature contours 120 when the applicator is inserted in living body tissue 122 and heating is caused by the means for radiating during operation of the applicator. The temperature sensor 118 is positioned at a point displaced in a distal direction from the means for radiating that corresponds to the approximate location of an outer margin of the expected heating area. The temperature sensor can be electrically connected to a connector 124 disposed at the distal end of the applicator, connected by a wire or wires 126. The conductive shell 110 (FIG. 1) can also function as an electrical connection to the temperature sensor. The temperature sensor can be left exposed to provide good thermal contact with the tissue into which it is inserted, or the temperature sensor can be covered by a thin protective material, such as heat shrink tubing.

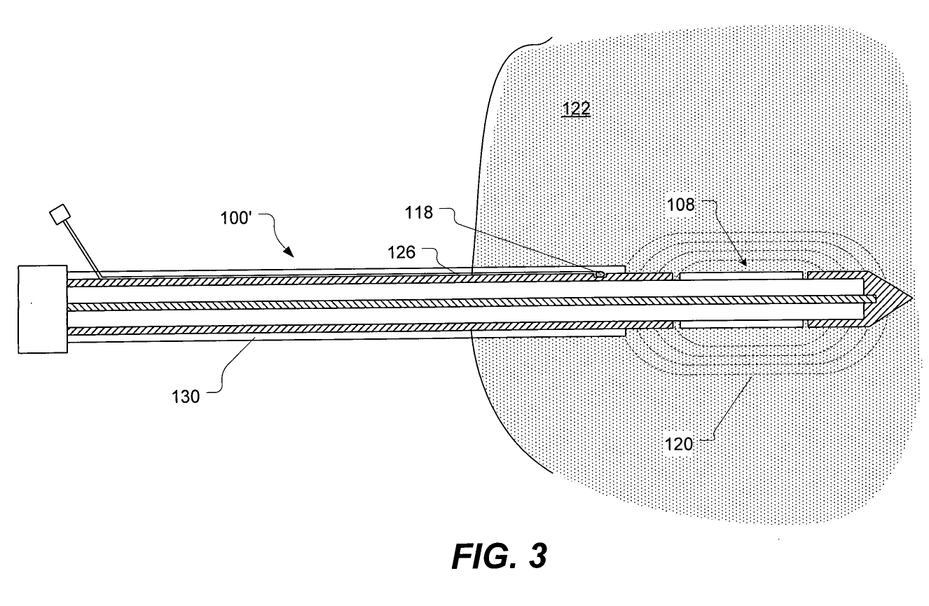

For example, FIG. 3 illustrates an alternate configuration of the microwave applicator 100′, which includes a sleeve 130 covering the temperature sensor 118 and it's connecting wires 126. The sleeve serves dual purposes, both helping to hold in place the sensor and helping to localize the radiated energy so that the radiated energy drops off quickly near the temperature sensor. For example, the sleeve can be a dielectric coating which helps to block energy that would otherwise tend to travel along the outside of the outer conductor. The sleeve can extend along the entire length of the applicator, and be made thinner over the antenna 108 than over the rest of the applicator to provide a similar effect. The dielectric shaping helps to cause a rapid attenuation of the heating pattern along the axis of the applicator when moving away from the antenna, resulting in less of a tear drop pattern. The higher the temperature of treatment is, the more helpful it becomes to have a rapid drop of energy at the boundary near the temperature sensor to avoid over heating of normal tissue along the inserted path.

When the microwave applicator 100 is used for heat treatment, the outer margin of the heating area will generally correspond to the boundary between diseased and healthy tissue. The temperature within the heating area will typically be higher than the perimeter. Thus, by placing the temperature sensor 118 at the outer margin of the heating area, temperature can be monitored at this important point. This can help to ensure that the healthy tissue is not damaged while also helping to ensure that the desired thermal dose is achieved within the tumor. For example, during treatment, temperature at the margin can be controlled to ensure that healthy tissue is not exposed to a thermal dose exceeding 200. Since temperatures inside the margin are generally higher, ensuring that the thermal dose at the margin approaches, but does not exceed, 200 provides confidence that adequate thermal dose has been applied to the diseased tissue.

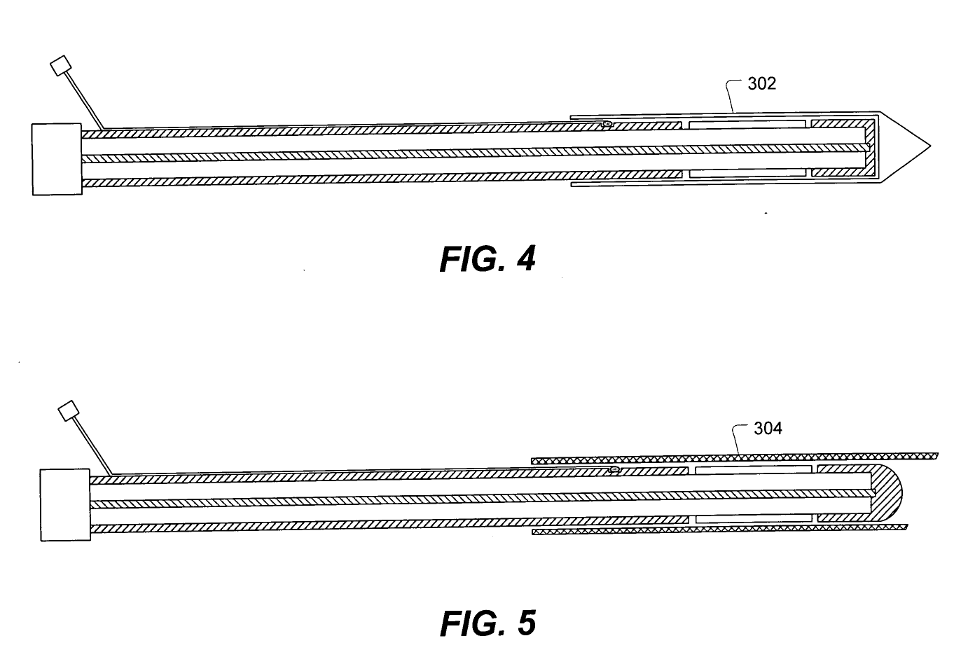

Continuing the discussion of the microwave applicator, the applicator 100 can include means for inserting the microwave applicator into a tissue region of the living body for invasive therapy. For example, as shown in FIG. 1, the microwave applicator can include a sharpened tip 126 at its proximal end 104 to enable direct insertion of the microwave applicator into the living body. Alternately, the microwave applicator can include a removable exterior sheath having a pointed or sharpened proximal end, in which case the tip need not be sharpened. For example, as shown in FIG. 4, the sheath can be a closed end catheter 302 having a sharpened tip. A closed end catheter can avoid the need to sterilize the applicator. Alternately, the sheath can be an open ended catheter or hollow needle. For example, as shown in FIG. 5, a metal hollow needle 304 can be used to insert the microwave applicator into living tissue, and then retracted or pulled back to expose the antenna during treatment. The sheath can be made of metal or plastic, and can be disposable. The simple construction of the microwave applicator and sheath helps to keep the cost of the applicator low, so that it can also be disposed of after use if desired.

Various configurations for the antenna 108 will now be described. As shown in FIG. 1, the antenna can be a dipole, where the center conductor 112 extends past a proximal end 114 of the outer conductor 110 to form the antenna. The tip 126 of the microwave applicator can be metal and electrically connected to the center conductor. An area between the end of the outer conductor and the tip can be left open, or can be filled with a dielectric material 128 to help provide stiffness to the microwave applicator. Microwave applicators can be designed having different length antennas to provide different size radiating regions. In other embodiments, the antenna can also include gaps in the outer conductor or include sleeves to improve the radiation pattern using techniques known in the art.

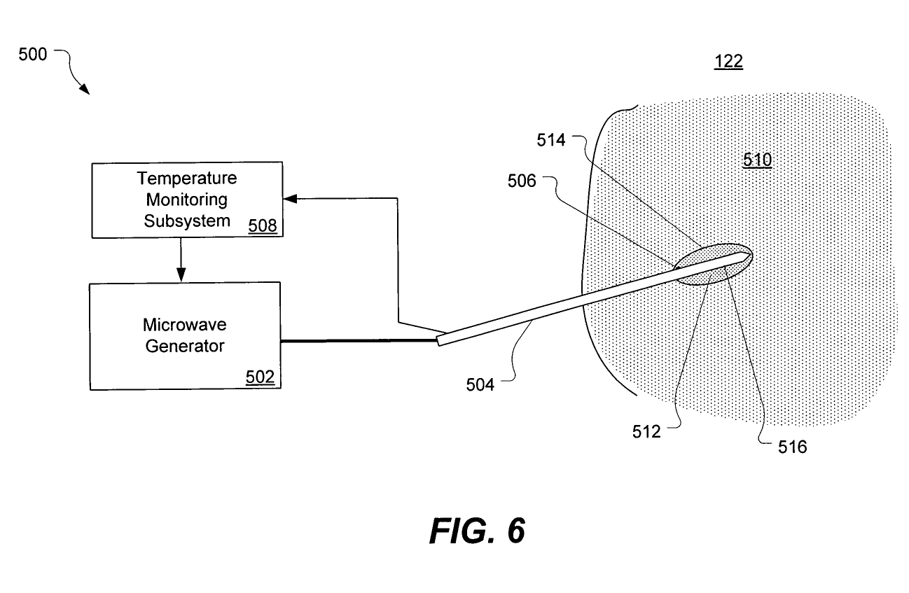

Use of the microwave applicator will now be described in conjunction with FIG. 6, which shows a block diagram of a system 500 for microwave therapy. The system includes a microwave generator 502 for outputting microwave energy. The microwave generator is coupled to a microwave applicator 504. The microwave applicator includes a temperature sensor 506, for example as described above. A temperature monitoring subsystem 508 is coupled to the temperature sensor, and adjusts the output of the microwave generator to maintain a desired temperature at the temperature sensor.

Therapy can also include using multiple microwave applicators 504 which are inserted into the living body tissue 510. Multiple applicators can allow larger or irregularly shaped areas to be heated while maintaining a more uniform heat distribution within the diseased tissue area 512. Generally, it is desirable to minimize the number of microwave applicators which are inserted into body tissue to help reduce trauma. In addition, it is desirable to maximize the uniformity of the power distribution within the treatment area. More uniform power distribution helps to provide more predictable temperature distributions which in turn results in better correspondence of the actual treatment to the prescribed treatment plan. Moreover, more uniform power distribution also helps to provide greater power efficiency of the power that enters the patient. Accordingly, pretreatment planning can be performed to optimize the number, size, and location of microwave applicators that will be used to help achieve these goals.

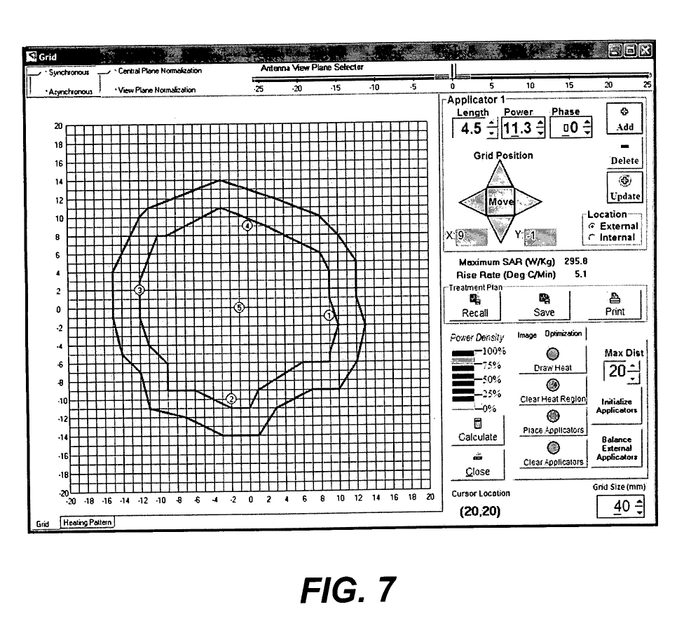

Pretreatment planning can include simulating a heating response of the living body tissue to applied microwave energy and determining a location for the microwave applicator(s) 504 that reduces heating outside the outer margin 514 of the diseased area and increases heating within the diseased area 512. Pretreatment planning can begin by obtaining a three-dimensional image of a tissue region within the living body. For example, magnetic resonance imaging (MRI) and similar techniques can provide three-dimensional images. A treating physician can then identify a three-dimensional target area within the image corresponding to the diseased area for which heat treatment is desired. For example, the diseased area can be indicated manually through a user interface to a computerized system by drawing outlines or shading the diseased area. Alternately, the diseased area may be automatically indicated using diagnostic algorithms programmed into a computer. For example, FIG. 7 illustrates a screen capture of an exemplary pretreatment planning system showing the ability to outline the target tumor area and showing placement of simulated applicators. Locations for insertion of the microwave applicators can also be specified, or automatically optimized by the simulation system. For example, once a simulation is performed, if the results do not correspond to the desired treatment plan, the locations can be adjusted to reduce heating outside the outer margin of the diseased area and increase heating within the outer margin of the diseased area.

The simulation can also take into account amplitudes and phases of the microwave energy applied to the applicators, since constructive and destructive interference will affect the distribution of heating. Accordingly, amplitude and phase settings can be determined to optimize the uniformity of heating within the diseased tissue and to minimize the amount of heating outside the diseased tissue. Simulation can also include accounting for different length radiating regions, for example, provided by microwave applicators having different antenna lengths. The simulation can be performed in three dimensions, allowing comparison of the predicted heating distribution to a desired distribution at all of the margins of the treatment area. For example, multiple two-dimensional slices of the simulated heating results can be obtained.

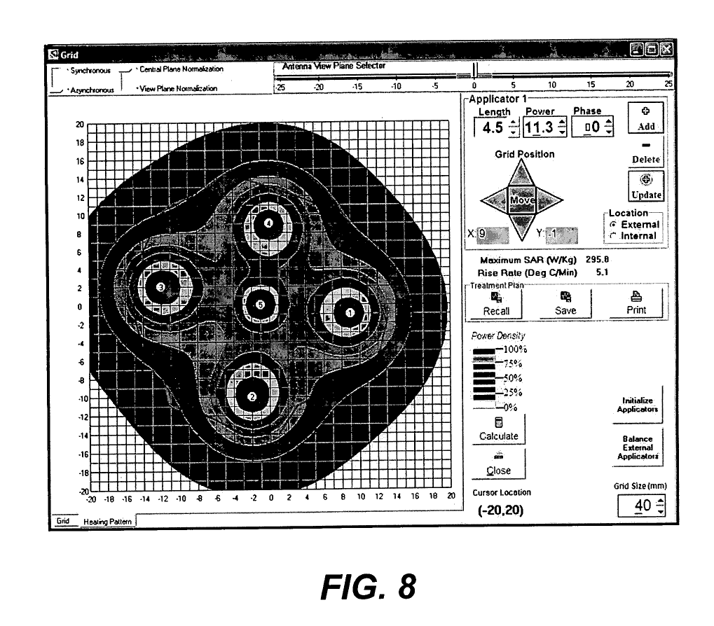

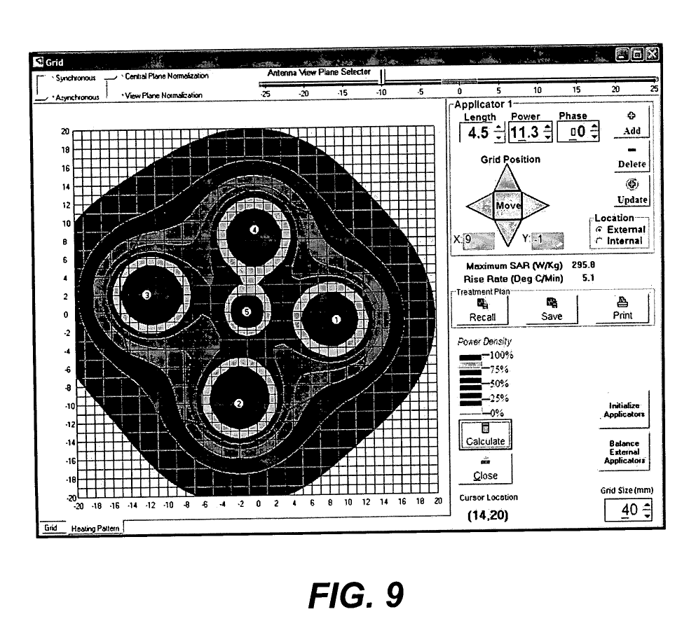

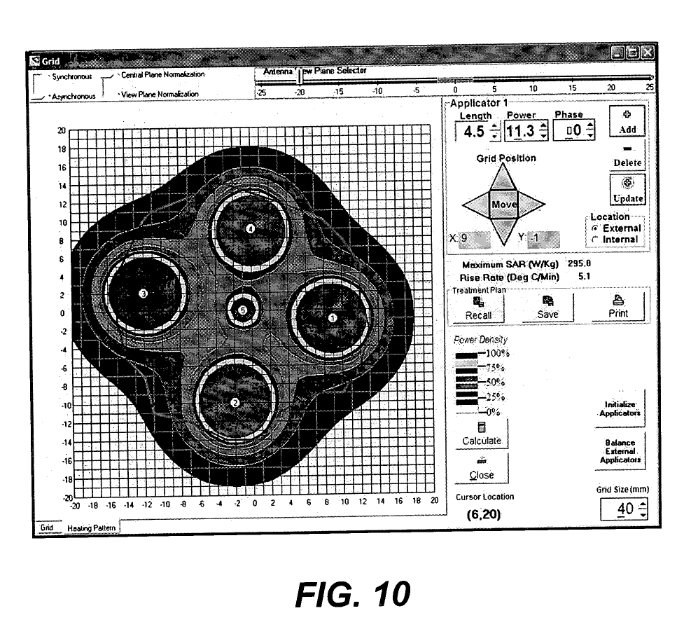

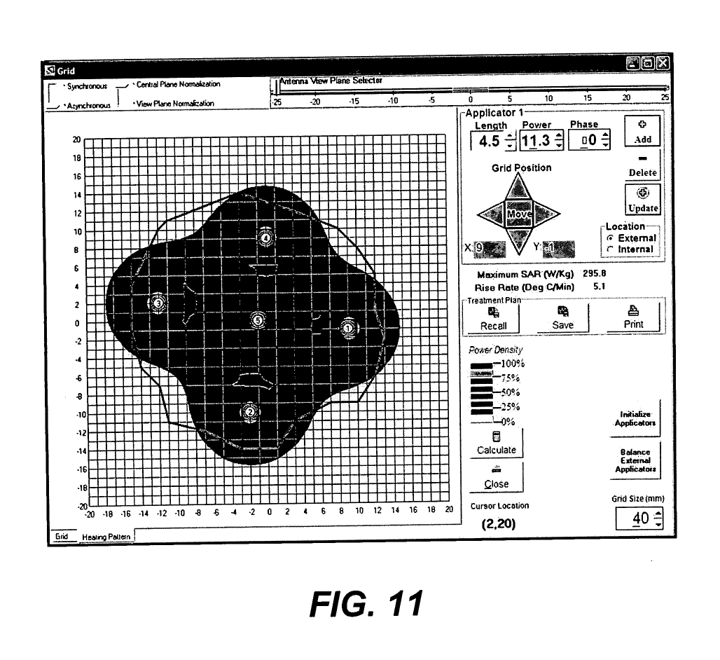

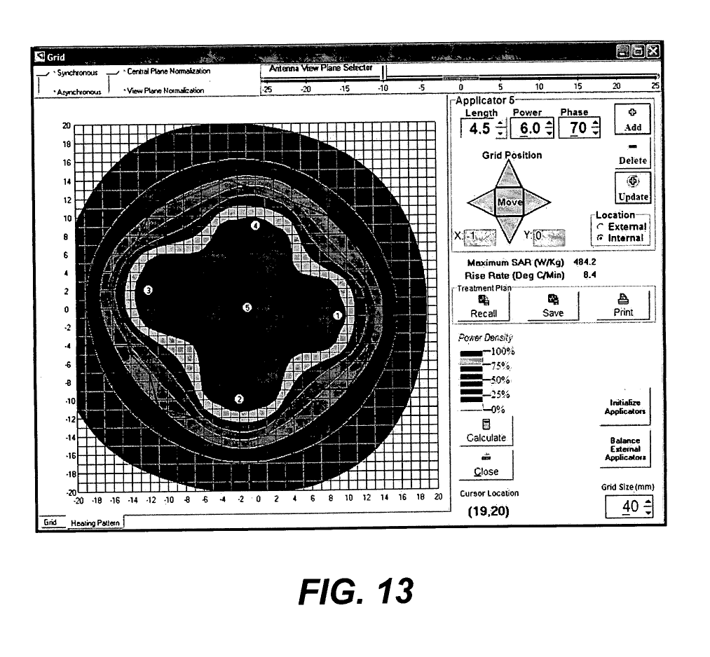

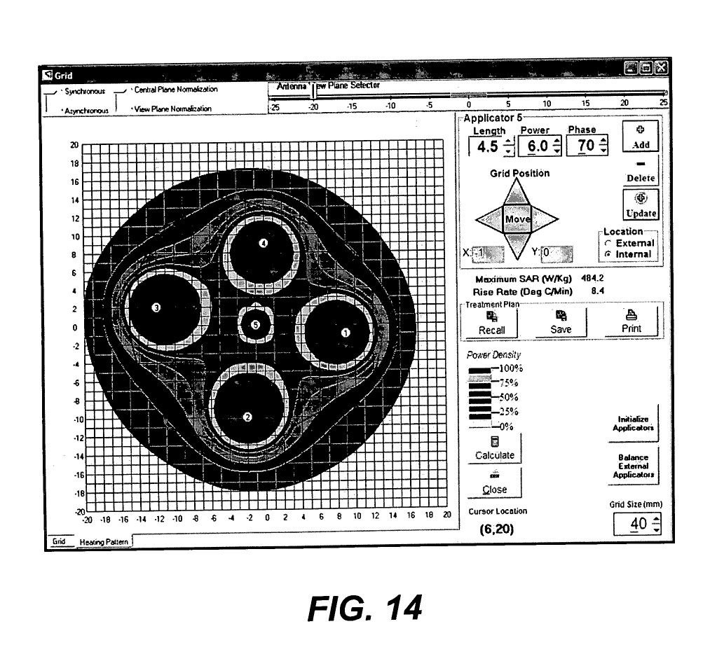

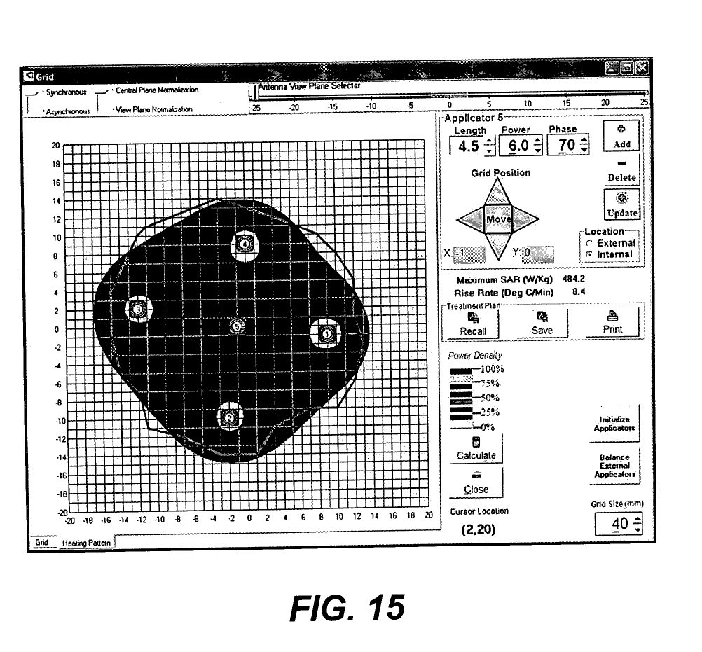

FIGS. 8-15 provide screen captures from an exemplary pretreatment planning system. The figures illustrate predicted heating patterns for an array of five applicators and a reference outline corresponding to the boundary of the tumor. FIGS. 8-11 illustrate a heating pattern for asynchronous phase operation. FIGS. 8-11 correspond to two-dimensional slices taken perpendicular to the applicator bodies, at positions offset by 0, 10, 20, and 25 mm from the center of the tumor. It can be seen that the heating pattern is non-uniform and corresponds poorly to the tumor shape. Accordingly, it is difficult in this example to ensure adequate heat treatment is applied to the tumor without causing damage to the surrounding healthy tissue.

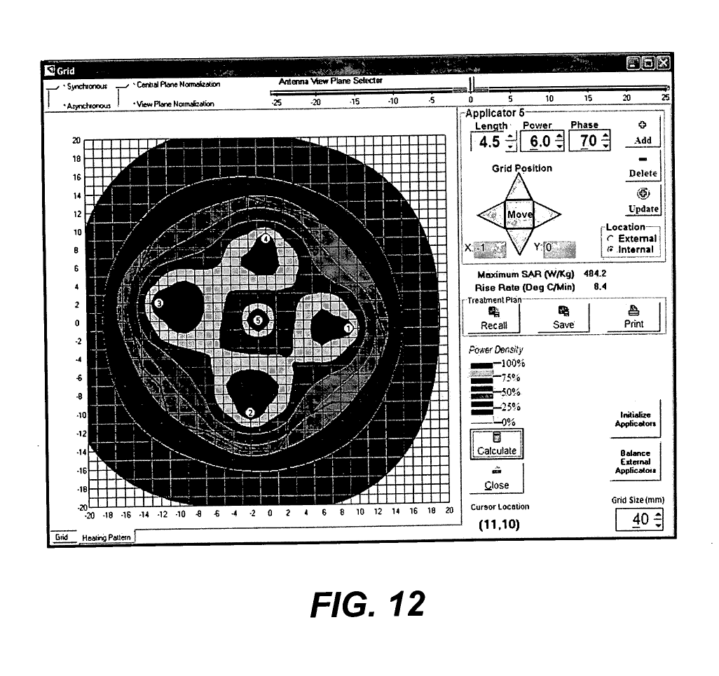

In contrast, FIGS. 12-15 provide illustrations of the heating pattern where the power and phase have been optimized. It can be seen that the heating power is more uniform, and corresponds more closely to shape of the tumor. For example, consider a treatment plan where the 100% power point is heated to cause the temperature to increase by 48 degrees C. above the normal body temperature of 37 degrees C., thus reaching a temperature of 85 degrees C. In such a case, the 12.5% contour would be raised by 6 degrees C. to a temperature of 43 degrees C. The 12.5% contour therefore represents a marginal temperature that will not cause tissue damage for treatment periods of up to 30 minutes. By using the simulation system, a number of different treatment plans can be tested and optimized before the actual procedure is performed on the patient.

The actual treatment procedure includes positioning one or more microwave applicators 504 into the living body tissue 510. Applicators may be selected to have a desired radiating region size (for example, specific lengths used during the pretreatment planning). The applicators are positioned so that the antenna 516 is inside the treatment area 512, and at least one temperature sensor 506 is positioned at an outer margin 514 of the diseased area. The locations can correspond to locations determined by pretreatment planning. When multiple applicators are used, multiple temperature sensors may be positioned at margins of the diseased area. Applying microwave energy to the microwave applicators causes radiation from the antenna, in turn causing heating within the diseased area.

The microwave generator 502 can include multiple outputs to allow application of amplitude and phase-controlled microwave energy to multiple applicators 504. The system can provide phase control using pre-calibrated phase shift modules or cable, in-line electronic phase shifters, and mechanically movable phase shifters such as ferrite and sliding length coaxial link stretchers, and the like. Amplitude control can be provided by attenuators, amplifiers, and the like. Phase and amplitude control can be provided externally to the microwave generator or included within the microwave generator.

The temperature monitoring subsystem 508 monitors the temperature at the temperature sensor(s) 506 and is used for feedback control of the applied power to maintain temperature at the desired level. During operation, deviations from the predicted heating distribution can be detected, and operation modified as necessary to more closely conform the heating to a prescribed treatment plan. Modification of the operation can include adjusting amplitude, phase, or terminating treatment. For example, treatment can be terminated when a desired temperature is reached at the outer margin of the diseased area. Alternately, the amount of microwave energy applied to the microwave applicator may be adjusted to maintain a desired temperature at the outer margin of the diseased area for a desired length of time.

In conclusion, the combination of pretreatment simulation and temperature monitoring at the margin of the diseased tissue provides better control over microwave heat therapy. Pretreatment simulation allows optimization in the number and location of invasive applicators which are inserted into the patient. Trauma can be reduced when fewer applicators are inserted. Three-dimensional simulation allows for more precise planning of the heating to be applied. More uniform heating can be obtained over an irregular region by specifying phase and amplitude distributions for the individual applicators. Monitoring of the temperature at the margin of the diseased tissue helps both to ensure that adequate heat is provided to the diseased tissue to meet the prescribed treatment plan and to ensure that heat application to nearby healthy tissue is limited to avoid damage to the healthy tissue. Accordingly, embodiments of the present invention may make heat treatment therapy a first line therapy for primary tumors such as prostate cancer and a preferable alternative to more aggressive and toxic treatments such as surgery, radiation, or chemotherapy by providing more uniform and adequate heating of the tumor to ensure that the tissue to be treated is heated to the required temperature and to avoid small areas of very high temperature, thereby possibly reducing excessive patient pain.

Whereas the invention is here illustrated and described with reference to embodiments thereof presently contemplated as the best mode of carrying out the invention in actual practice, it is to be understood that various changes may be made in adapting the invention to different embodiments without departing from the broader inventive concepts disclosed herein and comprehended by the claims that follow.

We claim:

1. A microwave applicator for insertion into living body tissue for heat treatment of diseased tissue within the living body, the microwave applicator comprising:
an elongate applicator body having a proximal end for insertion into a tissue region of the living body and a distal end for attachment to a source of microwave energy;
an antenna disposed toward the proximal end of the applicator body;
a microwave energy conductor disposed within the applicator body to conduct microwave energy from the distal end to the antenna;
a temperature sensor positioned along the applicator body so as to place the temperature sensor at a position corresponding to an outer margin of an expected heating area in the living body tissue caused by the antenna during operation of the applicator.

2. The microwave applicator of claim 1, wherein the temperature sensor is displaced in a distal direction from the antenna.

3. The microwave applicator of claim 1, wherein the microwave energy conductor is a coaxial transmission line and the elongate applicator body comprises a conductive shell to function as an outer conductor of the coaxial transmission line.

4. The microwave applicator of claim 3, wherein the conductive shell also functions as an electrical connection to the temperature sensor.

5. The microwave applicator of claim 3, wherein a center conductor of the coaxial transmission line extends past the conductive shell toward to the proximal end to form the antenna.

6. The microwave applicator of claim 5, wherein the antenna is a dipole.

7. The microwave applicator of claim 1, wherein the elongate applicator body comprises an exterior sleeve extending from the connector to a position on the proximal side of the temperature sensor so as to cover the temperature sensor.

8. The microwave applicator of claim 7, wherein the sleeve is a dielectric material so as to limit microwave energy propagating along an exterior of the microwave energy conductor.

9. The microwave applicator of claim 1, further comprising:
a first connector disposed at the distal end of the elongate applicator body and electrically coupled to the microwave energy conductor; and
a second connector disposed at the distal end of the elongate applicator body and in communication with the temperature sensor.

10. The microwave applicator of claim 1, further comprising a pointed tip at the proximal end to enable direct insertion of the microwave applicator into a tissue region of the living body.

11. The microwave applicator of claim 1, further comprising an exterior sheath having a pointing proximal end to enable direct insertion of the microwave applicator into a tissue region of the living body, the sheath being removable from the microwave applicator.

12. The microwave applicator of claim 1, further comprising an exterior sheath having an open end to enable the sheath to be retracted to expose the antenna during treatment.

13. A system for microwave therapy for heat treatment of diseased tissue within a living body, the system comprising:
a) a microwave generator for outputting microwave energy;
b) a microwave applicator coupled to the microwave generator, the microwave applicator having
i) an elongate applicator body having a proximal end for insertion into a tissue region of the living body and a distal end for attachment to a source of microwave energy;
ii) means for radiating microwave energy disposed toward the proximal end of the applicator body;
iii) means for conducting microwave energy disposed within the applicator body to conduct microwave energy output from microwave generator to the means for radiating microwave energy;
iv) means for sensing temperature at a position along the applicator body corresponding to an outer margin of an expected heating area in the living body tissue caused by the antenna during operation of the applicator;
c) a temperature monitoring subsystem coupled to the temperature sensor and the microwave generator and configured to adjust output of the microwave energy generator to maintain a desired temperature at the means for sensing temperature.

14. The system of claim 13 further comprising means for inserting the microwave applicator into a tissue region of the living body.

15. A method of microwave therapy for heat treatment of diseased tissue within a living body with a microwave applicator having an antenna and a temperature sensor, the method comprising the steps of:
obtaining a three-dimensional image of a tissue region within the living body;
identifying a three-dimensional target area within the image corresponding to a diseased area of the tissue region to be treated;
positioning the microwave applicator into the living body, so that the antenna of the microwave applicator is positioned inside the diseased area and the temperature sensor is positioned at an outer margin of the diseased area;
applying microwave energy to the microwave applicator to cause radiation from the antenna thereby causing heating within the diseased area; and
monitoring temperature at the outer margin of the diseased area with the temperature sensor.

16. The method of claim 15, further comprising the steps of:
simulating a heating response of the living body to applied microwave energy; and
determining a location for the microwave applicator that reduces heating outside the outer margin of the diseased area and increases heating within the diseased area.

17. The method of claim 15, further comprising the step of selecting a microwave applicator having a desired radiating region size.

18. The method of claim 15, further comprising the step of positioning a plurality of microwave applicators into the living body, so that the antenna of each microwave applicator is positioned inside the diseased area and at least one temperature sensor is positioned at an outer margin of the diseased area.

19. The method of claim 15, further comprising terminating treatment when a desired temperature is reached at the outer margin of the diseased area.

20. The method of claim 15, further comprising controlling the amount of microwave energy applied to the microwave applicator to maintain a desired temperature at the outer margin of the diseased area for a desired length of time.

21. The method of claim 15, wherein the step of positioning the microwave applicator into the living body is aided by an exterior sheath.
